(19) United States
(12) Patent Application Publication
(10) Pub. No.: US 2015/0100084 A1
(43) Pub. Date: Apr. 9, 2015

(54) DEVICES AND METHODS FOR SUTURING

(71) Applicant: Boston Scientific Scimed, Inc., Maple Grove, MN (US)

(72) Inventors: Claude O. CLERC, Marlborough, MA (US); Laurie A. SODERBOM, Nashua, NH (US)

(21) Appl. No.: 14/507,020

(22) Filed: Oct. 6, 2014

Related U.S. Application Data
(60) Provisional application No. 61/888,126, filed on Oct. 8, 2013.

(51) Int. Cl. A61B 17/06; A61B 17/04

(52) U.S. Cl. CPC A61B 17/06166; A61B 17/06066; A61B 17/0401; A61B 2017/061; A61B 2017/06176
USPC 606/223; 606/228; 606/230

(57) ABSTRACT

The present disclosure generally relates to medical devices including sutures and methods of use thereof. The medical device may include a suture having projections angled with respect to a longitudinal axis of the suture and a needle coupled to the suture for inserting the suture into tissue, wherein the projections may allow movement of the suture in a first direction and prevent movement in a second direction. The suture may include an anchor at a distal end such that once the needle is removed, the suture may be pulled in a proximal direction to engage the anchor with an inner tissue wall to tighten and secure the suture within the tissue.

### CROSS-REFERENCE TO RELATED APPLICATIONS

This application claims the benefit of priority from U.S. Provisional Application No. 61/888,126, filed on Oct. 8, 2013, the entirety of which is incorporated herein by reference.

### TECHNICAL FIELD

The present disclosure generally relates to medical devices and methods of use thereof for performing medical procedures. More particularly, the present disclosure relates to sutures and related devices and methods for joining tissue, such as securing the stomach to the abdominal wall in a gastropexy procedure.

### BACKGROUND

In some medical procedures, it may be necessary or useful to fixedly connect a portion of tissue to another. When creating a stoma for placement of an enteral feeding device, such as a gastric, jejunal, transgastric jejunal, or percutaneous endoscopic gastrostomy (PEG) tube, for example, the stomach may be attached to the abdominal wall in a gastropexy procedure to facilitate placement of the tube for enteral feeding. Attaching the stomach to the abdominal wall with sutures may allow adhesions to form between the two tissues so that the attachment remains after the sutures are absorbed or removed. Thus, the attachment may facilitate initial placement of the feeding tube as well as dilation of the stoma tract. Attaching the tissues together also may facilitate replacement of the feeding tube, which is typically done every 3-6 months. Further, attaching the stomach to the abdominal wall may help to prevent inadvertent displacement of the feeding tube, or exposure of the peritoneal cavity to contamination leading to health risks for the patient. Exposure to contamination may occur if the feeding tube migrates into the peritoneal cavity, for example, creating a serious health risk if nutrition is administered through the feeding tube while it is in the peritoneal space.

Sutures may need to remain in place for 2-3 weeks while the stoma site forms, or longer to ensure the feeding tube remains secure. Sutures often include external anchors to keep them in place, or require tying suture ends together or to an external fastening point such as the feeding tube or the patient's skin. Such anchors can be a source of discomfort or irritation for the patient at the stoma site, and can also increase the complexity of installing sutures.

### BRIEF SUMMARY

Embodiments of the present disclosure generally relate to sutures and related devices and methods of use thereof.

The present disclosure includes a method of performing a medical procedure, the method comprising: inserting a suture into an abdomen and through a wall of a stomach, the suture having a longitudinal axis and including multiple projections along a length of the suture and angled in a first direction with respect to the longitudinal axis, the suture including an anchor at a distal end of the suture such that the anchor is located inside the stomach; and moving the suture in a proximal direction to engage the anchor with the wall of the stomach, the plurality of projections allowing movement of the suture in the proximal direction while preventing movement in an opposite, distal direction; wherein moving the suture in the proximal direction shortens a length of the suture extending from an interior surface of the wall of the stomach to an exterior surface of a wall of the abdomen.

Embodiments of the present disclosure may include one or more following features, including combinations of the following features: inserting the suture may include inserting a needle coupled to the suture into the abdomen, wherein the needle may include a lumen that receives the suture; the needle may be coupled to a tube, the tube including a lumen that receives the suture; the method may comprise removing the needle before moving the suture in the proximal direction; the suture may include a polymer material; the suture may include a bioabsorbable material; the suture may inhibit movement of the stomach wall relative to the wall of the abdomen; only a proximal portion of the suture may include the plurality of projections; the first direction may point toward an interior of the stomach; the anchor may include an elongate bar for engaging with the interior surface of the stomach wall; the suture may be a first suture, the method further comprising repeating the inserting step and the moving step for a second suture to secure the second suture at a location adjacent to the first suture.

The present disclosure further includes a method of performing a medical procedure, the method comprising inserting a first needle and a second needle into an abdomen and through a wall of a stomach, wherein each of the first and second needles includes a lumen, the lumen of the first needle including a suture such that a distal end of the suture is located inside the stomach; wherein the suture includes a first section of projections angled in a first direction with respect to a longitudinal axis of the suture and a second section of projections angled in a second direction with respect to the longitudinal axis opposite to the first direction, a proximal end of the suture including the first section of projections and the distal end of the suture including the second section of projections; and moving the distal end of the suture into the lumen of the second needle and in a proximal direction outside the abdomen; removing the first and second needles such that each of the first and second sections of projections engage with the wall of the stomach; and moving each of the proximal and distal ends of the suture in the proximal direction, wherein each of the first and second sections of projections allows movement of the suture in the proximal direction and prevents movement of the suture in an opposite, distal direction; wherein moving the suture in the proximal direction shortens a length of the suture extending from an interior surface of the wall of the stomach to an exterior surface of a wall of the abdomen.

Embodiments of the present disclosure may include one or more following features, including combinations of the following features: the second needle may include a grasper within the lumen of the second needle, the method may further comprise using the grasper to grasp the distal end of the suture and move the suture in the proximal direction through the lumen of the second needle; at least a portion of the suture may include a bioabsorbable material; a portion of the suture between the proximal end and the distal end may not include projections.

The present disclosure further includes a medical device comprising at least one suture that may having a longitudinal axis, the suture including a first section of projections angled in a first direction with respect to a longitudinal axis of the suture and a second section of projections angled in a second direction with respect to the longitudinal axis opposite to the first direction, a proximal end of the suture including the first section of projections and a distal end of the suture including the second section of projections; and at least one needle, wherein the at least one needle may include a lumen for receiving the at least one suture.

Embodiments of the present disclosure may include one or more of the following features, including combinations of the following features: the at least one suture may include a portion between the proximal end and the distal end that does not include projections; the at least one suture may include a bioabsorbable material; the at least one needle may include a first needle and a second needle, each of the first and second needles including a lumen; or a grasper receivable within the lumen of the first needle or the second needle.

### BRIEF DESCRIPTION OF THE DRAWINGS

Embodiments of the present invention are described with reference to the following drawings for exemplary purposes only, wherein like reference numerals refer to like parts throughout unless otherwise specified. Reference will be made to the following Detailed Description, which is to be read in association with the accompanying drawings, wherein:

FIG. 1 illustrates an exemplary suture, according to an embodiment of the present disclosure;

FIG. 2 illustrates an exemplary suture and needle, in accordance with an embodiment of the present disclosure;

FIG. 3 illustrates another exemplary suture and needle, in accordance with an embodiment of the present disclosure;

FIG. 4 illustrates another exemplary suture, in accordance with an embodiment of the present disclosure;

FIG. 5 illustrates another exemplary suture and needles, in accordance with an embodiment of the present disclosure; and

FIG. 6 illustrates another exemplary suture, in accordance with an embodiment of the present disclosure.

### DETAILED DESCRIPTION

Embodiments of the present disclosure include sutures, devices, and related methods of use for performing a medical procedure such as gastropexy. In some embodiments, the device may be used to secure the abdominal wall to the stomach without the need for an external anchoring mechanism, such as an external anchor (e.g., a clip or button) or tying ends of a suture together, to a feeding tube, or to a patient's skin. Embodiments of the present disclosure may reduce and/or eliminate the need for an external anchoring mechanism for securing a suture, which may improve patient comfort and/or facilitate or otherwise improve a method of performing a gastropexy procedure, e.g., by reducing the complexity or number of steps involved in the procedure. Reducing and/or eliminating the need for an external anchoring mechanism may also improve safety for the patient, e.g., by reducing the choking hazard of an external anchor separated from a bioabsorbable suture. Choking hazards may be especially dangerous in pediatric patients.

In the following sections, embodiments of the presently disclosed subject matter will be described with respect to performing gastropexy for exemplary purposes. It will be understood that the present disclosure may also be used in other suitable medical procedures. For example, the present disclosure may be useful for cystopexy, sigmoidopexy, or any other procedure requiring the securement together of tissue walls.

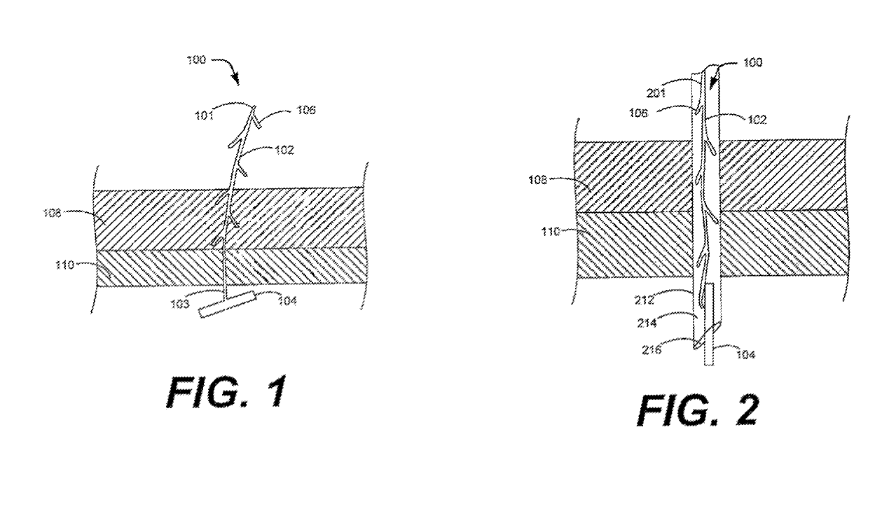

FIG. 1 shows an exemplary suture 100 joining an abdominal wall 108 to a stomach wall 110 for a gastropexy procedure. During gastropexy, the suture 100 may be introduced and/or implanted within a patient's body to secure the abdominal wall 108 to the stomach wall 110, such as during implantation of a PEG tube.

The suture 100 may include an elongated shaft 102 having a proximal end 101, a distal end 103, and a length extending therebetween. In general, the suture 100 may be sufficiently flexible to be navigated through tissue without causing injury. It should be noted that the flexibility of the suture 100 may be either uniform or may vary along its length. For example, the suture 100 may be more rigid adjacent the distal end 103 relative to the proximal end 101, e.g., to facilitate introduction into a patient's tissue. The proximal end 101 of the suture 100 may be configured to remain external to the patient's body while the distal end 103 may be configured to be introduced in the patient's body.

The suture 100 may include a plurality of projections 106 or barbs, e.g., forming a quill-type structure. While FIG. 1 shows six projections 106, any number of projections 106 can be disposed along a longitudinal length of the suture 100. In some embodiments, the suture 100 may include only one projection 106. The projections 106 may be equidistant from each other or may vary in distance along the length of the shaft 102. The distance between adjacent projections 106 may vary, for example, in accordance with a particular embodiment or application of the suture 100.

The projections 106 may be configured to hold and/or grasp tissue upon implantation into the body. For example, the projections 106 may be arranged along the length of the elongated shaft 102 at one or more angles with respect to the shaft 102 such that the suture 100 may be moved in only one direction. That is, the angle of the projections 106 may allow for movement of the suture 100 in a first direction and inhibit or otherwise prevent movement in a second direction, e.g., as the projections 106 engage with tissue. The projections 106 may extend radially outwards from the longitudinal axis of the suture 100, e.g., towards the distal end 103 of the suture 100 as shown in FIG. 1. Thus, the suture 100 may secure the surrounding tissue in such a manner that the suture 100 may move in a proximal direction towards the body surface but may not move in the distal direction towards the interior of the body.

The projections 106 may be arranged along the shaft 102 in any suitable manner. For example, FIG. 1 shows all of the projections 106 pointing generally in the same direction, towards the distal end 103 of the suture 100. In other embodiments, one or more projections may point in a different direction with respect to another projection, as shown in FIG. 4 (discussed below).

Further, the suture 100 may include projections 106 disposed at the same or different longitudinal position of another projection 106. FIG. 1 shows the projections 106 at different longitudinal positions from one another along the length of the shaft 102, e.g., two rows of projections 106 arranged on opposite sides or about 180° apart about the circumference of the shaft 102. In other embodiments, two or more projections may be disposed at the same longitudinal position (see, e.g., FIGS. 4-6, discussed below). For example, the suture 100 may include projections 106 arranged about 180° apart, about 120° apart, about 90° apart, or about 45° apart about the circumference of the shaft 102 for the same longitudinal position. In some embodiments, the projections 106 may be arranged in a spiral configuration along the length of the elongated shaft 102.

The projections 106 may generally be of the same length extending radially outward from the shaft 102, or may vary in length. For example, one or more projections 106 near a distal end 103 of the suture 100 may be longer in length than one or more projections 106 near a proximal end of the suture 100, or vice-versa. It should be noted that any of the above configurations of projections 106 may be combined in a single suture 100.

Further referring to FIG. 1, the suture 100 may include an anchor 104 operably coupled to the distal end 103 of the elongated shaft 102. In at least one embodiment, the anchor 104 may include a longitudinal bar. As shown in FIG. 1, for example, the anchor 104 and the shaft 102 of the suture 100 may form a T-shaped structure. Other suitable configurations for the anchor 104 may be used such as a tab, cross, spring, malecot, or button.

The anchor 104 may be used to tighten the suture 100, e.g., shorten a length of the suture 100 within the tissue when pulling on the suture 100 to approximate two or more layers of tissue to be joined. For example, FIG. 1 shows the anchor 104 located along an interior surface of the stomach wall 110. The projections 106 may be configured to permit the suture 100 to be pulled proximally through both the abdominal wall 108 and stomach wall 110 such that the distance or length of the suture 100 located within the abdominal wall 108 and/or stomach wall 110 may be shortened while the anchor 104 remains located inside the stomach. The projections 106 also may prevent the suture 100 from moving distally back through the tissue, and thus secure the suture 100 within the tissue.

The suture 100 may comprise any suitable biocompatible material, natural or synthetic. For example, the suture 100 may include one or more polymers or copolymers, fibers, metals or metal alloys, or any combination thereof. In some embodiments, the suture 100 may include one or more bioabsorbable and/or biostable materials such that the suture 100 or one or more portions thereof may be dissolved within and/or absorbed by the patient's body after a period of time. Exemplary bioabsorbable materials include polymers or copolymers such as polyglycolic acid (PGA), e.g., Vicryl (Ethicon, Inc.), polylactic acid (PLA), poly (lactic-co-glycolic) acid, polydioxanone (PDS), poly(glycolide-caprolacone), e.g., Monocryl (Ethicon, Inc.), or polycaprolactone.

In some embodiments, the suture 100 may include two or more different materials. For example, the anchor 104 may include a relatively rigid material in comparison to the shaft 102. In some embodiments, at least a portion or the entire shaft 102 may include a bioabsorbable material whereas the anchor 104 may include a non-bioabsorbable material such that the shaft 102 may dissolve or be absorbed after a period of time, leaving only the anchor 104. In other embodiments, the anchor 104 may include a bioabsorbable material, or a mixture of bioabsorbable and non-bioabsorbable materials. The remaining anchor 104 may be released into the stomach to pass through and outside of the body via the gastroenterological system. The suture 100 or a portion thereof may include one more coating(s) on an outer surface, such as a lubricating coating or an anti-bacterial coating.

The suture 100 may be implanted into the body with a needle as illustrated in FIG. 2, wherein the suture 100 is disposed within a lumen 214 of a needle 212. The suture 100 may be slidably disposed within the lumen 214 such that the suture 100 may move in a distal direction through a distal opening 216 of the needle. The needle 212 may comprise any suitable biocompatible material(s) such as one or more metals, metal alloys, polymers, polymer composites, or glass. The needle 212 may be configured to puncture tissue, e.g., with a sharp distal end 216.

The anchor 104 may be aligned substantially parallel to the longitudinal axis of the suture 100 when disposed within the lumen 214 of the needle 212. Once the needle 212 reaches a target location in the body (e.g., when the distal opening 216 of the needle 212 extends beyond an inner wall or surface of tissue such as beyond the abdominal and stomach walls 108, 110), the needle 212 may be proximally withdrawn with respect to the suture 100 to release the suture 100 in the tissue of the abdominal wall 108 and stomach wall 110. Once released from the needle 212, the anchor 104 may be aligned substantially perpendicular to the longitudinal axis of the suture 100, e.g., to engage with an inner surface of the stomach wall 110. It should be noted that the anchor 104 may self-align against the stomach wall 110 (e.g., via shape memory alloy or other elastic action) or may be manipulated via a proximal actuation mechanism to form a T-shaped or other suitable configuration for engaging with the stomach wall 110. Although not shown, the needle 212 and/or suture 100 may be coupled proximally to an actuation mechanism to facilitate introduction, navigation, and/or implantation of the needle 212 and/or the suture 100. Exemplary actuation mechanisms may include a wire-based mechanism and/or a spring-based mechanism. In some embodiments, for example, the actuation mechanism may include a push-wire to hold the anchor 104 in place during delivery and push it into the stomach 110 after introduction of the needle 212.

Once released from the needle 212, the suture 100 may be moved in a proximal direction, e.g., pulled from outside the abdominal wall 108 to engage the anchor 104 with the inner wall of the stomach 110. The projections 106 may allow movement of the suture 100 in the proximal direction while preventing movement in the opposite, distal direction. Moving the suture 100 in the proximal direction may shorten the length of the suture 100 located within the tissue and increase tension with the tissue to secure the suture 100 in place without the need of an anchor external to the body.

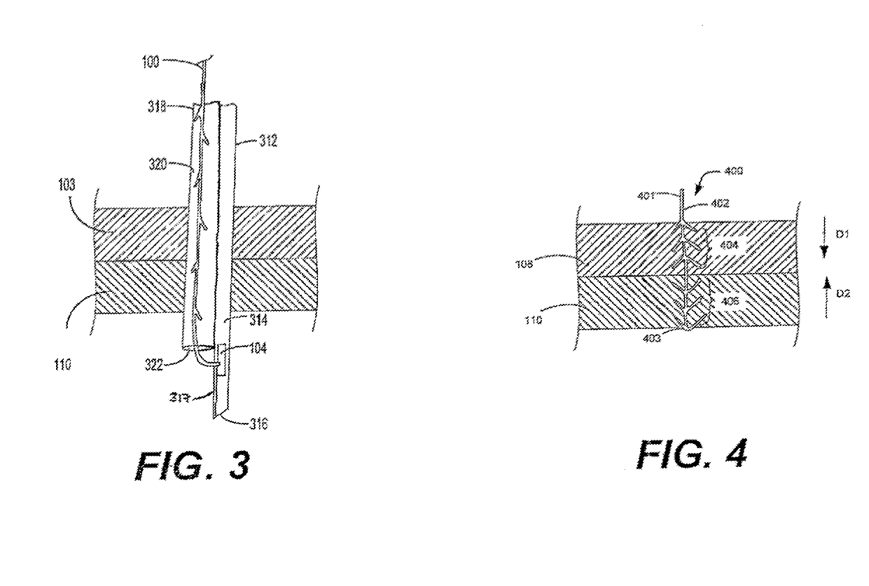

In some embodiments, the suture 100 may be inserted into the body via medical device comprising a tube coupled to a needle. As shown in FIG. 3, the suture 100 may be slidably disposed within a lumen 320 of a tube 318 coupled to a needle 312. The needle 312 may include any of the features of the needle 212 of FIG. 2. The anchor 104 may be placed inside a lumen 314 of the needle 312 and slidable through a distal opening of the needle 316 via a slot 317 in the wall of the needle 312. It should be noted that, in other embodiments, the anchor 104 may remain within the lumen 320 of the tube 318 with the rest of the suture 100.

To implant the suture 100, the needle 312 and the tube 318 carrying the suture 100 may be inserted into the body at the same time. For example, as the needle 312 punctures the abdominal wall 108, it may carry the tube 318 and suture 100 in a distal direction through the abdominal and stomach walls 108, 110. In other embodiments, the needle 312 and tube 318 may be inserted into the tissue together first, followed by insertion of the suture 100 through the lumen 320 of the tube 318.

Once a target site in the tissue is reached, the anchor 104 may be pushed distally through the slot 317 and released from the needle 312. The needle 312 then may be withdrawn with the tube 318 to release the suture 100 in the tissue. In some embodiments, the needle may be withdrawn before or after withdrawal of the tube 318. As discussed above, the suture 100 then may be pulled in a proximal direction to engage the anchor 104 against an inner wall of the stomach 110 to tighten the suture 100.

The tube 318 may include any suitable biocompatible material(s) such as, but not limited to, metals, metal alloys, or polymers (e.g., plastic or silicone). In one embodiment, the tube 318 may include a relatively soft and/or flexible material in comparison to the needle 312. The needle 312 therefore may provide an overall strength to the medical device, while facilitating navigation through the patient's body. While FIG. 3 shows a generally circular tube 318, devices with other shapes and cross-sections may be used to insert the suture 100 alongside the needle 312.

Turning now to FIG. 4, another exemplary suture 400 may include a first set or section of projections 404 and a second set of projections 406, each disposed along an elongate shaft 402 of the suture 400. The suture 400 of FIG. 4 may include any of the features of suture 100 of FIGS. 1-3. The first set of projections 404 may be disposed at or near a proximal end 401 of the suture 400, and the second set of projections 406 may be disposed at or near distal end 403 of the suture 400. The first and second sets of projections 404, 406, each may include any of the features discussed above with respect to projections 106 of FIGS. 1-3.

As shown in FIG. 4, the first set of projections 404 may point towards the distal end 403 of the suture 400 (indicated by directional arrow DO, while the second set of projections 406 may point towards the proximal end 401 (indicated by directional arrow D2). Since the two sets of projections 404 and 406 are arranged in opposing directions, pulling the suture 400 proximally may only move the portion of the suture 400 with projections 404, thus shortening the length of the suture 400 in the abdominal wall 108 only. The direction D2 of the projections 406 may prevent the suture 400 from moving proximally within the wall of the stomach 110. Thus, in some embodiments, the suture 400 may not include an internal anchor, thus avoiding potential complications associated with an internal anchor (e.g., the anchor becoming embedded in the tissue wall) and any secondary procedures to address such complications.

While FIG. 4 shows the first set of projections 404 entirely within the stomach wall 110 and the second set of projections 406 entirely within the abdominal wall 108, in other embodiments, either set of projections 404, 406 may extend through multiple tissue walls. The angle formed by each projection 404, 406 and the longitudinal axis of the suture 400 may be the same or different with respect to any other projection(s) 404, 406. In some embodiments, a length of the elongate shaft 402 between the first set and second set of projections 404, 406 may not include any projections. Further, embodiments of the present disclosure may include more the two sets of projections, e.g., three, four, five or more set of projections.

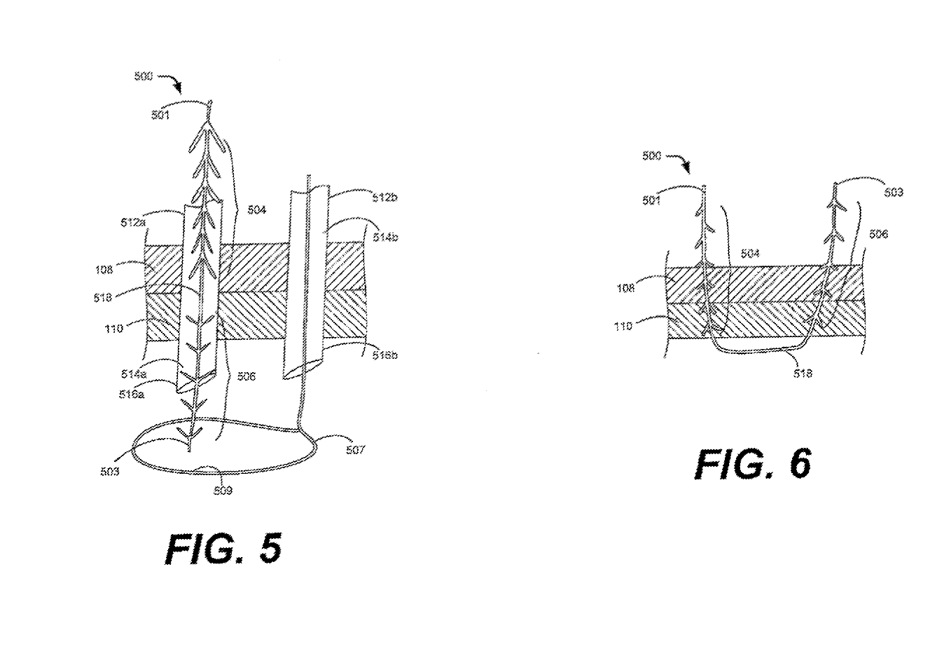

FIGS. 5 and 6 show another exemplary suture 500, which may include any of the features of sutures 100 and/or 400 discussed above. As shown, the suture 500 extends from a proximal end 501 to a distal end 503, and includes a first set of projections 504 and second set of projections 506 similar to projections 404, 406 of FIG. 4. In some embodiments, the suture 500 may be implanted into the body using more than one needle, for example two needles. FIG. 5 shows a first needle 512a and second needle 512b disposed within the abdominal wall 108 and stomach wall 110 such that a distal end of each needle (516a and 516b, respectively) extends beyond the inner surface of the stomach wall 110. The first and second needles 512a, 512b may be aligned substantially parallel and next to each other.

The suture 500 may be slidably disposed within a lumen 514a of the first needle 512a. The second needle 512b may include a grasper 507 slidably disposed within a lumen 514b and configured to grasp the distal end 503 of the suture 500 while disposed within the first needle 512a. The grasper 507 may be actuated by a proximal actuation mechanism to grasp the suture 500. For example, the grasper 507 may be configured to move between a collapsed configuration (e.g., while disposed within the lumen 514b of the second needle 512b) and an expanded configuration (e.g., once advanced distally outside the lumen 514b). For example, the grasper 507 may be actuated by a push-pull mechanism such that the grasper 507 may be pushed distally beyond the distal end of the second needle 514b to move from the collapsed configuration to the expanded configuration. In some embodiments, the grasper 507 may be self-expandable for grasping the suture 500.

The grasper 507 may have a loop 509 or snare-shaped structure as shown in FIG. 5. Other suitable grasping structures may be used, such as a basket, clamp, or forceps. The grasper 507 may be manipulated such that the diameter of the loop 509 substantially encircles the distal end 503 of the suture 500, e.g., once the suture 500 is advanced distally through the first needle 514a or the first needle 514a is suitably withdrawn to expose the distal end 503 of the suture 500.

Once the loop 509 encircles the distal end 503 of the suture 500 (or the distal end 503 of the suture 500 is aligned to rest within the loop 509), the loop 509 may be tightened around the suture 500, e.g., by reducing the diameter of the loop 509. The grasper 507 may then be withdrawn proximally through the lumen 514b of the second needle 512b to carry the distal end 503 of the suture outside the body, e.g., outside the abdomen wall 108 at a location adjacent the entry point of the suture 500. The distal end 503 of the suture 500 may then be pulled while the first and second needles 512a, 512b remain in place such that first set of projections 504 may be disposed within the lumen 514a of the first needle 512b and the second set of projections 506 may disposed within the lumen 514b of the second needle 512b. A middle portion 518 of the suture 500 between the first and second sets of projections 504, 506 not including any projections may remain within the stomach.

The first and second needles 512a, 512b then may be removed such that the first and second sections of projections 504, 506 engage with the tissue of the abdominal and stomach walls 108, 110, as shown in FIG. 6. The proximal and distal ends 501, 503 of the suture 500 each may be pulled in the proximal direction such that each of the first and second sets of projections 504, 506 allows movement of the suture 500 in the proximal direction and prevents movement of the suture in the opposite, distal direction. This may tighten or otherwise secure the suture 500, e.g., by shortening the lengths of the suture 500 extending from the inner stomach wall 110 to the outer abdomen wall 108. In some embodiments, the same or different needles 512a, 512b, may be used for the implantation or placement of a second suture, which may be similar to the first suture 500, and may be adjacent to or otherwise in proximity to the first suture 500.

Multiple sutures as disclosed herein may be used in a given procedure, and any of the methods discussed herein may be repeated, e.g., to secure multiple sutures at multiple locations. In some embodiments, the method may include the placement of two, three, four, five, or more sutures, in any configuration. For example, three sutures may be placed in a triangular configuration, allowing for the creation of a stoma in the triangle, such as at or near the center of the triangle. Further, different types of sutures may be used in a single procedure, e.g., any combination of sutures 100, 400, 500, or other variations and/or modifications thereof may be used together. Further, the present disclosure may include use of commercial devices such as the Easy Tie™ device (Boston Scientific, Natick, Mass.), the Funada Loop Fixture II gastropexy device (Create Medic Co., Ltd., Yokohama, Japan), the Cope Gastrointestinal Anchor Set (Cook Medical, Bloomington, Ind.), or the Saf-T-Pexy device (Kimberly-Clark, Irving, Tex.).

While the embodiments described above are generally directed to sutures for gastropexy, the present disclosure is not so limited and may be applicable to other tissues and medical devices used for attaching tissue. Moreover, while specific embodiments may have been illustrated and described collectively herein, any subsequent arrangement designed to achieve the same or similar purpose may be substituted for the specific embodiments described and shown herein. Features from each of the embodiments can be used or interchanged with any of the features of the other embodiments. Combinations of the above embodiments, and other embodiments not specifically described herein, will be apparent to those of skill in the art upon reviewing the description.

It is intended that the specification and examples be considered as exemplary only, and departure in form and detail may be made without departing from the scope and spirit of the present disclosure as defined by the following claims.

What is claimed is:

1. A method of performing a medical procedure, the method comprising:
inserting a suture into an abdomen and through a wall of a stomach, the suture having a longitudinal axis and including a plurality of projections along a length of the suture and angled in a first direction with respect to the longitudinal axis, the suture including an anchor at a distal end of the suture such that the anchor is located inside the stomach; and
moving the suture in a proximal direction to engage the anchor with the wall of the stomach, the plurality of projections allowing movement of the suture in the proximal direction and preventing movement in an opposite, distal direction;
wherein moving the suture in the proximal direction shortens a length of the suture extending from an interior surface of the wall of the stomach to an exterior surface of a wall of the abdomen.

2. The method of claim 1, wherein inserting the suture includes inserting a needle coupled to the suture into the abdomen, wherein the needle includes a lumen that receives the suture.

3. The method of claim 2, wherein the needle is coupled to a tube, the tube including a lumen that receives the suture.

4. The method of claim 2, further comprising removing the needle before moving the suture in the proximal direction.

5. The method of claim 1, wherein the suture includes a polymer material.

6. The method of claim 1, wherein the suture includes a bioabsorbable material.

7. The method of claim 1, wherein the suture inhibits movement of the stomach wall relative to the wall of the abdomen.

8. The method of claim 1, wherein only a proximal portion of the suture includes the plurality of projections.

9. The method of claim 1, wherein the first direction points toward an interior of the stomach.

10. The method of claim 1, wherein the anchor includes an elongate bar for engaging with the interior surface of the stomach wall.

11. The method of claim 1, wherein the suture is a first suture, the method further comprising repeating the inserting step and the moving step for a second suture to secure the second suture at a location adjacent to the first suture.

12. A method of performing a medical procedure, the method comprising:
inserting a first needle and a second needle into an abdomen and through a wall of a stomach, wherein each of the first and second needles includes a lumen, the lumen of the first needle including a suture such that a distal end of the suture is located inside the stomach;
wherein the suture includes a first section of projections angled in a first direction with respect to a longitudinal axis of the suture and a second section of projections angled in a second direction with respect to the longitudinal axis opposite to the first direction, a proximal end of the suture including the first section of projections and the distal end of the suture including the second section of projections;
moving the distal end of the suture into the lumen of the second needle and in a proximal direction outside the abdomen;
removing the first and second needles such that each of the first and second sections of projections engage with the wall of the stomach; and
moving each of the proximal and distal ends of the suture in the proximal direction, wherein each of the first and second sections of projections allows movement of the suture in the proximal direction and prevents movement of the suture in an opposite, distal direction;
wherein moving the suture in the proximal direction shortens a length of the suture extending from an interior surface of the wall of the stomach to an exterior surface of a wall of the abdomen.

13. The method of claim 12, wherein the second needle includes a grasper within the lumen of the second needle, the method further comprising using the grasper to grasp the distal end of the suture and move the suture in the proximal direction through the lumen of the second needle.

14. The method of claim 12, wherein at least a portion of the suture includes a bioabsorbable material.

15. The method of claim 12, wherein a portion of the suture between the proximal end and the distal end does not include projections.

16. A medical device comprising:
at least one suture having a longitudinal axis, the suture including a first section of projections angled in a first direction with respect to a longitudinal axis of the suture and a second section of projections angled in a second direction with respect to the longitudinal axis opposite to the first direction, a proximal end of the suture including the first section of projections and a distal end of the suture including the second section of projections; and
at least one needle, wherein the at least one needle includes a lumen for receiving the at least one suture.

17. The medical device of claim 16, wherein the at least one suture includes a portion between the proximal end and the distal end that does not include projections.

18. The medical device of claim 16, wherein the at least one suture includes a bioabsorbable material.

19. The medical device of claim 16, wherein the at least one needle includes a first needle and a second needle, each of the first and second needles including a lumen.

20. The medical device of claim 19, further comprising a grasper receivable within the lumen of the first needle or the second needle.
